(19) United States
(12) Patent Application Publication
(10) Pub. No.: US 2022/0168197 A1
(43) Pub. Date: Jun. 2, 2022

(54) Powder System For The Care Of Hair

(71) Applicant: Henkel AG & Co. KGaA, Duesseldorf (DE)

(72) Inventors: Marlene Battermann, Asendorf (DE); Sylvia Kerl, Boenningstedt (DE)

(21) Appl. No.: 17/539,148

(22) Filed: Nov. 30, 2021

(30) Foreign Application Priority Data
Nov. 30, 2020 (DE) 102020215082.6

(51) Int. Cl. A61K 8/26; A61Q 5/12; A61K 8/73; A61K 8/02; A61K 8/67; A61K 8/55; A61K 8/34; A61K 8/46; A61K 8/44

(52) U.S. Cl. CPC A61K 8/26; A61Q 5/12; A61K 8/732; A61K 8/0241; A61K 2800/48; A61K 8/553; A61K 8/345; A61K 8/466; A61K 8/442; A61K 8/678

(57) ABSTRACT

A cosmetic composition in powder form for the care of keratinous fibres, which contains specific amounts of at least one surfactant, at least one binder and/or thickener and at least one hair-conditioning active ingredient, and which is dissolved and foamed in contact with water. The present invention further relates to a method of conditioning hair using the powder cosmetic composition.

### FIELD OF THE INVENTION

The application describes cosmetic agents in powder form for the care of keratinous fibres based on surfactants, binding and/or thickening agents as well as hair-conditioning active ingredients which can be dissolved and foamed in contact with water. The application further describes methods for the preparation of the powders and their use for hair care.

### BACKGROUND OF THE INVENTION

Hair care products have been known for a long time and are offered in the trade, for example, as conditioners and/or hair treatments in liquid or paste form in suitable packaging. End users take the required amount of product from the packaging during use and dispose of it after emptying. Such products offer the user the advantage of simple and quick handling, which is why they dominate the market today. However, this advantage is achieved by accepting certain disadvantages, which are discussed below. In most cases, the packaging of the described products in liquid or paste form consists of non-recyclable plastic, which is a serious problem from an environmental point of view in view of the constantly growing amount of plastic waste.

Another problem is that previous products usually contain higher quantities of water or water/solvent mixtures, which means that the products have a larger volume and, possibly of greater importance from a transport point of view, a relatively high weight. This is disadvantageous for several reasons. In times of increasing water scarcity, resources should be saved. An undesirable, increased transport volume associated with large-volume heavy products is also important from an environmental and cost perspective. Another interesting point is that worldwide travel activity is constantly increasing. Consumers are therefore increasingly interested in cosmetic products that are easy to transport due to their low weight and volume. This is particularly relevant with regard to air travel, as larger containers containing liquids are generally excluded from being carried in an aircraft cabin, so that a passenger travelling only with hand luggage often finds himself in the situation, due to the cosmetics products that dominate the market today, of not being able to take his preferred product selection with him or having to decant the corresponding products into smaller containers first, which, however, is generally accompanied by an even greater volume of packaging material.

### BRIEF SUMMARY OF THE INVENTION

The provision of alternative product forms with lower water content, which are contained in more environmentally friendly, for example recyclable, packaging to save space, is therefore an important goal in the formulation of improved, contemporary and sustainable cosmetic products.

WO 2019/023332 discloses shampoos in powder form containing substantially sulphate-free surfactants (and other surfactants) and thickening and conditioning agents. The disadvantage of these shampoos is their strong foaming capacity, which is rather undesirable in hair care products.

There is therefore still a need for hair care products in powder form, which, in addition to the aforementioned requirements, also have a conditioning benefit on hair.

In view of the problems and requirements described above, the inventors have therefore set themselves the task of providing cosmetic hair care formulations in powder form which can be produced in an energy-saving manner and are easy to use, and in which the use of preservatives can essentially be dispensed with.

The formulations in powder form should have a pleasant, creamy consistency after dissolution in water and should not foam strongly. In addition, the powders should have an increased conditioning benefit on the hair.

### DETAILED DESCRIPTION OF THE INVENTION

The task of the invention is solved by the formulations, methods and uses described in detail below.

Cosmetic composition in powder form for the care of keratinous fibres, containing, by weight of the total composition
a) 5.0 to 35.0% by weight of at least one surfactant,
b) 40.0 to 80.0% by weight of at least one binder and/or thickener,
c) 0.1 to 15.0% by weight of at least one hair conditioning active ingredient.

A powder-form care composition according to the preceding point, comprising—based on the total weight of the care composition—6.0 to 34.0% by weight, preferably 7.0 to 33.0% by weight and particularly preferably 8.0 to 32.0% by weight of at least one surfactant a).

A powder-form care composition according to one of the preceding points, containing as surfactant(s) a) anionic surfactants, preferably amino acid-based surfactants, isethionates and/or alkyl sulphates.

A powder-form care composition according to one of the preceding points, containing as surfactant(s) a) non-ionic, cationic, amphoteric and/or zwitterionic surfactants.

Care composition in powder form according to one of the preceding points, containing binders and/or thickeners b) of vegetable and/or mineral origin in a proportion by weight of at least 95% (based on the total weight of all binders and/or thickeners in the powder).

Powdery care composition according to one of the preceding points, containing as binder and/or thickener b) clay minerals and/or polysaccharides.

Powdery care product according to the preceding point, containing bentonites, starches and/or starch derivatives.

A powder-form care composition according to one of the preceding points, containing—based on the total weight of the care composition—20.0 to 60.0% by weight, preferably 25.0 to 55.0% by weight and particularly preferably 30.0 to 50.0% by weight of at least one starch and/or a starch derivative.

A powdered care composition according to one of the preceding points, containing starch fractions from maize, potatoes, rice, wheat, tapioca and/or dextrins, amylose, amylopectin, and/or hydroxypropyl starch phosphate.

A powdered care composition according to one of the preceding points, containing—based on the total weight of the care composition—10.0 to 40.0% by weight, preferably 15.0 to 35.0% by weight and particularly preferably 20.0 to 30.0% by weight of bentonite.

Powder-form care composition according to one of the preceding points, containing at least one, preferably at least two and particularly preferably at least three hair-conditioning active ingredient(s) c) from the groups of the
Refatting agents and/or lipids,
Emollients,
Moisturiser,
Vitamins, vitamin derivatives and/or vitamin precursors,
cationic polymers and/or
Anti-dandruff active ingredients.

A powdered care composition according to any one of the preceding points, which is substantially free from water and/or preservatives.

Care product in powder form according to one of the preceding points, which is dissolved and/or emulsified in contact with water.

Process for the preparation of a powdered care product according to any one of the preceding points, in which all the ingredients are mixed and optionally sieved in a mixing apparatus for cosmetic powder products.

Cosmetic process for hair care, in which a powdered cosmetic care product according to one of the preceding points is dissolved in water, rubbed between the hands and/or emulsified, distributed on the hair and rinsed off with water after an exposure time of 10 seconds to 15 minutes.

Cosmetic use of a cosmetic care composition in powder form according to one of the preceding points for the care of the hair, in particular for the improvement of the
Hair feel, in particular the hair feel on wet hair and/or in the lengths and/or ends of the hair,
Disentanglability and/or
Combability.

Accordingly, a first object of the present invention is a cosmetic composition in powder form for the care of keratinous fibres, comprising, based on the total weight of the composition
a) 5.0 to 35.0% by weight of at least one surfactant,
b) 40.0 to 80.0% by weight of at least one binder and/or thickener,
c) 0.1 to 15.0% by weight of at least one hair conditioning active ingredient.

The previously defined powdered cosmetic hair care products offer the right properties for single-application portions, especially in terms of their dissolution retention and spread ability on the hair during use. The high concentrations of active substances in the powders are associated with the advantages of using few resources in their production and transportation, and that they can be easily transported without great effort or restrictions, whether to the gym or on an airplane trip.

When mixed with water, the powders according to the invention transform into an emulsion with an excellent feel, which foams little to not at all, and which can be distributed very well in the hair and rinsed out of it again.

In addition, the application of the powdered hair care compositions according to the invention significantly improved important care parameters such as hair feel, detangling properties and/or combability.

The powder according to the invention comprises, as a first essential ingredient, from 5.0 to 35.0% by weight of at least one surfactant a) (based on the total weight of the powder).

Preferably, hair care compositions according to the invention comprise certain surfactant contents which leave the hair and/or scalp feeling pleasant without drying out or stressing the hair. The surfactant content should be high enough (even after mixing the powder in/with water immediately before application) to achieve sufficient effect and at the same time low enough not to foam up too much—preferably not at all.

In a first preferred embodiment, the powdered hair care composition contains—based on its total weight—6.0 to 34.0% by weight, preferably 7.0 to 33.0% by weight and particularly preferably 8.0 to 32.0% by weight of at least one surfactant a).

Suitable surfactants a) can be selected from the group of anionic surfactants, for example:
a) Ether carboxylic acids of the formula R—O—(CH₂—CH₂O)ₓ—CH₂—COOH, in which R is a linear or
   is a branched, saturated or unsaturated alkyl group with 8 to 30 C atoms and x=0 or 1 to 16,
b) surfactants available from natural sources such as
   Acylglycinates,
   Acylsarcosinates,
   Acyl lactylates,
   Acylglutamates,
   Acylaspartates,
   Acyltaurates,
   Acyl isethionates,
   Sulphosuccinates and
      the corresponding salified forms of the above-mentioned surfactants, the acyl groups comprising from 10 to 30 carbon atoms,
c) Alpha-olefin sulfonates with 8 to 24 C atoms (alpha-olefin sulfonate surfactants,
d) Alkyl sulphates of the formula R—O—SO₃X, in which R is preferably a straight or branched chain,
   is a saturated or unsaturated alkyl group with 8 to 30 C atoms and X is an alkali metal, alkaline earth metal, ammonium or alkanol amine ion,
e) Sulfonates of unsaturated fatty acids with 8 to 24 carbon atoms and 1 to 6 double bonds
f) Esters of tartaric acid and citric acid with alcohols which are addition products of about 2-15 molecules of ethylene oxide and/or propylene oxide onto fatty alcohols containing 8 to 22 carbon atoms, and/or
g) Alkyl and/or alkenyl ether phosphates of the formula

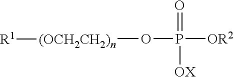

in which R¹ is preferably an aliphatic hydrocarbon radical having 8 to 30 carbon atoms, R² is hydrogen, a radical (CH₂CH₂O)ₙR¹ or X, n is numbers from 0 to 10 and X is hydrogen, an alkali metal or alkaline earth metal or the group —NR³R⁴R⁵R⁶, where R³ to R⁶ independently of one another are a C₁ to C₄ hydrocarbon radical.

Particularly preferred are surfactants from the aforementioned group b), as well as their corresponding salified forms, in which the acyl groups comprise 10 to 24 carbon atoms and in particular 10 to 18 carbon atoms.

Examples of particularly suitable surfactants from group b) are alkali metal, alkaline earth metal and/or alkanolamine salts of so-called amino acid-based surfactants such as Cocoyl Glycinate, Cocoyl Sarcosinate, Lauroyl Sarcosinate, Myristoyl Sarcosinate, Oleyl Sarcosinate, Cocoyl Glutamate, Lauroyl Glutamate, Stearoyl Glutamate, Lauroyl Aspartate, Palmitoyl Aspartate, (C₁-C₄-Alkyl) Cocoyl Taurate, (_{C1-C4 alkyl}) Lauroyl Taurate, (C₁-C₄-Alkyl) Oleyl Taurate as well as Lauroyl Lactylate, Stearoyl Lactylate, (C₁-C₄-Alkyl) Cocoyl Isethionate, (C₁-C₄-Alkyl) Lauroyl Isethionate, Lauryl Sulfosuccinate and/or any mixtures thereof.

Acylglutamates and/or acyl isethionates are particularly preferred.

Furthermore, surfactants from group d) are particularly preferred, because C₈-C₃₀ alkyl sulphates are very well tolerated by the skin.

C₁₀-C₂₄ alkyl sulphates are highly preferred, and C₁₂-C₁₈ alkyl sulphates are particularly preferred, because these anionic surfactants, also known as “coco sulphates,” are obtained from natural coconut oil and are particularly suitable as ECOCERT-compliant ingredients for use in the powders of the invention.

Alkali salts such as potassium or sodium salts of coco sulphates are particularly preferred. Sodium Coco Sulphate (SCS) is particularly preferred.

Anionic surfactants from groups b) and d) have proved to be particularly suitable for providing skin-compatible and skin- and hair-caring powders according to the invention.

Amino acid-based surfactants, isethionates and/or alkyl sulphates are particularly preferred.

In a second particularly preferred embodiment, powders according to the invention contain anionic surfactant(s) a) from the group of amino acid-based surfactants, isethionates and/or alkyl sulphates.

Within this embodiment, acylglutamates, isethionates and/or alkyl sulphates are particularly preferred because they are mild and skin-friendly and they impart improved combability, smoothness, softness and shine to hair. Furthermore, acylglutamates, isethionates and/or alkyl sulphates can be produced on a purely vegetable basis and are also suitable for vegan cosmetics.

In a further preferred embodiment, powders according to the invention contain—based on their total weight.
ai) 10 to 30% by weight of at least one acyl glutamate and
aii) 0 to 10% by weight of at least one isethionate.

These concentration ranges have proven to be particularly suitable. When working with these concentration ranges, the powders have balanced properties in terms of application feel and care performance.

Suitable surfactants a) can also be selected from the groups of non-ionic, cationic, amphoteric and/or zwitterionic surfactants.

These surfactants can be used both individually and as a mixture with the aforementioned anionic surfactants in the hair care compositions according to the invention.

Suitable amphoteric and/or zwitterionic surfactants in the sense of the present invention are Alkyl betaines, alkylamidoalkyl betaines, alkylamphoacetates, alkylamphodiacetates, alkylamphopropionates, alkylamphodipropionates, alkylsultaines, alkylhydroxysultaines, alkylamine oxides, alkylamphoglycinates, alkyliminodiacetates, alkyliminodipropionates, alkylamphopropylsulphonates, alkylamphocarboxyglycinates and alkylamphocarboxypropionates.

Suitable alkyl betaines and/or alkylamidopropyl betaines preferably comprise C₄-C₂₄, more preferably C₆-C₁₈, particularly preferably C₈-C₁₄ alkyl chains, which may be linear or branched, linear chains being preferred.

Cocamidopropyl betaine is particularly preferred.

Particularly suitable alkylamphoacetates, alkylamphodiacetates, alkylamphopropionates or alkylamphodipropionates are the compounds known under the INCI names sodium cocoamphoacetate and disodium cocooamphodiacetate.

The hair care compositions in powder form according to the invention may contain amphoteric and/or zwitterionic surfactants in a proportion by weight of 0 to 10% (more preferably 0.1 to 8.0%, particularly preferably 0.2 to 6.0% and especially 0.3 to 5.0%) of the total weight of the cosmetic hair care composition.

Suitable non-ionic surfactants in the sense of the present invention are:
Amine oxides, for example the surfactants known under the INCI names

Cocamine Oxide, Lauramine Oxide and/or Cocamidopropylamine Oxide and commercially available from various suppliers,
Fatty acid alkanolamides, in particular the compounds known under the INCI designation Cocoamide MEA,
Fatty alcohol alkoxylates (especially ethoxylates) with a C-chain length of 8 to 24, especially 10 to 20, and a degree of alkoxylation (ethoxylation degree) of 2 to 30, such as laureth-4,
Mixtures of alkyl(oligo)glucosides and fatty alcohols, for example, the commercially available product Montanov®68,
Alkyl(oligo)glycoside. Suitable alkyl (oligo)glycosides can be selected from compounds of the general formula of RO-[G]x, in which [G] is preferably derived from aldoses and/or ketosis with 5-6 carbon atoms, preferably from glucose.

The index number x stands for the degree of oligomerization (DP), i.e. the distribution of mono- and oligoglycosides. The index number x preferably has a value in the range from 1 to 10, particularly preferably in the range from 1 to 3, whereby it may not be an integer but a fractional number that can be determined analytically.

Particularly preferred alkyl (oligo)glycosides have a degree of oligomerization between 1.2 and 1.5. The radical R preferably represents at least one alkyl and/or alkenyl radical containing 4 to 24 carbon atoms.

Especially preferred alkyl (oligo)glycosides are the compounds known under the INCI designations Caprylyl/Capryl Glucosides, Decyl Glucosides, Lauryl Glucosides and Coco Glucosides.

The hair care compositions in powder form according to the invention may contain non-ionic surfactants in a proportion by weight of from 0 to 5.0% by weight (more preferably from 0.1 to 4.0% by weight, particularly preferably from 0.2 to 3.0% by weight and especially from 0.3 to 2.0% by weight) of the total weight of the cosmetic hair care composition.

In principle, all cationic surface-active substances suitable for use on the human body are suitable as cationic surfactants in compositions according to the invention. These are characterized by at least one water-solubilizing cationic group, such as a quaternary ammonium group, or by at least one water-solubilizing cationizable group, such as an amine group, and further by at least one lipophilic alkyl group having about 6 to 30 C atoms, or also by at least one imidazole group or at least one imidazylalkyl group.

In general, cationic surfactants are divided into groups according to their structural characteristics. Particularly suitable for use in the powdered compositions according to the invention are cationic surfactants a) from at least one of the groups of alkylquats, esterquats, quaternary imidazolines, amidoamines and/or cationized amidoamines.

These specifically named cationic surfactants a) have shown a conditioning effect in the compositions according to the invention that is perceived as particularly pleasant.

Particularly preferred compositions according to the invention contain as cationic surfactants a) quaternary ammonium compounds (alkylquats) having at least one C₈-C₂₄ alkyl radical, esterquats and amidoamines each having at least one C₈-C₂₄ acyl radical, and mixtures thereof.

Quaternary ammonium compounds with at least one C₈-C₂₄ alkyl radical are particularly preferred ammonium halides, especially chlorides, and ammonium alkyl sulphates, such as methosulphates or ethosulphates, such as C₈-C₂₄ alkyl trimethylammonium chlorides, C₈-C₂₄ dialkyldimethylammonium chlorides and C₈-C₂₄ trialkylmethylammonium chlorides, e.g. cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as the imidazolium compounds known under the INCI designations Quaternium-27, Quaternium-83, Quaternium-87 and Quaternium-91. The alkyl chains of the surfactants mentioned above preferably have 8 to 24 carbon atoms.

Esterquats are cationic surfactants which contain both at least one ester function and at least one quaternary ammonium group as a structural element and furthermore at least one C₈-C₂₄ alkyl radical or C₈-C₂₄ acyl radical. Preferred esterquats are quaternized ester salts of fatty acids with triethanolamine, quaternized ester salts of fatty acids with diethanolalkylamines and quaternized ester salts of fatty acids with 1,2-dihydroxypropyldialkylamines. Such products are sold under the trademarks Stepantex®, Dehyquart® and Armocare®. N,N-Bis(2-palmitoyloxyethyl)dimethylammonium chloride, distearoylethyl dimonium methosulfates, distearoylethyl hydroxyethylmonium methosulfates and bis-(isostearoyl/oleoyl isopropyl) dimonium methosulfates are preferred examples of such esterquats.

Bis-(Isostearoyl/oleoyl isopropyl) dimonium methosulphate is a particularly preferred esterquat.

The alkylamidoamines are usually produced by amidation of natural or synthetic C₈-C₂₄ fatty acids and fatty acid sections with di-(C₁-C₃)alkylaminoamines. Compounds from this substance group which are particularly suitable according to the invention are for example the compounds known under the INCI designations stearamidopropyl dimethylamine, behenamidopropyl dimethylamine and/or brassicamidopropyl dimethylamine. Stearamidopropyl dimethylamine is particularly preferred.

The hair care compositions in powder form according to the invention may contain cationic surfactants in a proportion by weight of from 0 to 15.0% by weight (more preferably from 0.1 to 12.5% by weight, particularly preferably from 0.2 to 10.0% by weight and especially from 0.3 to 7.5% by weight) of the total weight of the cosmetic hair care composition.

In a third preferred embodiment, the powdered hair care compositions according to the invention contain as surfactant(s) a) non-ionic, cationic, amphoteric and/or zwitterionic surfactants.

Within this embodiment, it is particularly preferred if the hair care compositions contain non-ionic, cationic, amphoteric and/or zwitterionic surfactants in addition to previously mentioned anionic surfactants.

The powder according to the invention comprises, as a second essential ingredient, 40.0 to 80.0% by weight of at least one binder and/or thickener b) (based on the total weight of the powder).

For the provision of sustainable products, it is advantageous if the binding and/or thickening agents as the main component of the powder according to the invention are predominantly derived from natural sources.

In a fourth preferred embodiment of the invention, the powdered care compositions therefore contain binders and/or thickeners b) of vegetable and/or mineral origin in a proportion by weight of at least 95% (based on the total weight of all binders and/or thickeners in the powder).

Suitable binders and/or thickeners b) of vegetable and/or mineral origin within the meaning of the present invention may preferably be selected from clay minerals and/or polysaccharides.

For the purposes of the present invention, binding and/or thickening agents b) of mineral origin are preferably understood to be clay minerals. The term clay mineral refers on the one hand to minerals that occur predominantly in the finest grains (grain size <2 μm), but on the other hand to the phyllosilicates, which are named after their layered crystal structure consisting of silicon and oxygen, as well as hydrogen and mostly magnesium and aluminium. The two definitions are not congruent.

It is preferred that the clay mineral is selected from the group consisting of the kaolin group, the smectite group, the illite-mica group, the chlorite group and mixtures thereof.

Preferably, the clay mineral is a phyllosilicate selected from the group consisting of kaolinite, halloysite, dickite, nacrite, dioctahedral vermiculite, dioctahedral illite, dioctahedral smectite, montmorillonite, beidellite, nontronite, volkonskoite, nacrite, trioctahedral vermiculite, trioctahedral smectite, saponite, hectorite, sauconite and mixtures thereof.

Clay minerals can be added to the remedy in the form of clays. There are different clays, which are characterised by a different composition of clay minerals and possibly layer silicates. Clay comes in white, pink, red-yellow, brown and green. The colour of green clay is due to copper compounds. In pink, red and brown clay, iron is the colouring element. White clay, which is also called kaolin, refers to a fine, iron-free, white clay that contains kaolinite as its main component. Halloysite, dickite and nacrite also occur. It may also be preferred that bentonite is added as a clay mineral. Bentonite is a clay. Bentonite is a mixture of different clay minerals and contains montmorillonite (60% to 80%) as its most important component.

Bentonite is a particularly preferred clay mineral in the sense of the present invention.

Natural polysaccharide-based binding and/or thickening agents b) within the meaning of the present invention are preferably water-soluble or water-swellable polysaccharides which are preferably composed of more than ten monosaccharide units and are obtainable in powder form.

Preferred polysaccharides are
the celluloses and/or starches composed of glucose units and their derivatives and/or degradation products such as amylose, amylopectin and dextrins. Chemically and/or thermally modified starches, e.g. hydroxypropyl starch phosphate, dihydroxypropyl distarch phosphate or the commercial products Dry Flo®, are also advantageous according to the invention. Dextranes and their derivatives, e.g. dextran sulphate, are also preferred. Also preferred are non-ionic cellulose derivatives, such as methylcellulose, hydroxypropylcellulose, hydroxypropylmethylcellulose or hydroxyethylcellulose;
the polysaccharides composed of fucose units, e.g. the commercial product Fucogel®;
the inulins composed of fructose units and a terminal glucose residue;
Heteroglycans such as alginic acid, carob gum, carrageenan, gum arabic, pectins, tragacanth, xanthan gum.

Particularly preferred are starch fractions from corn, potatoes, rice, wheat and/or tapioca and/or derivatives of starch such as amylose, amylopectin, dextrin and/or hydroxypropyl starch phosphate.

In a fifth preferred embodiment, the powdered hair care compositions according to the invention contain b) clay minerals and/or polysaccharides as binders and/or thickeners.

Within this embodiment, it is particularly preferred if the hair care compositions contain bentonites, starches and/or starch derivatives as clay minerals and/or polysaccharides.

Quite particularly preferred within the fifth preferred embodiment are powdered hair care compositions according to the invention which contain—based on their total weight—20.0 to 60.0% by weight, preferably 25.0 to 55.0% by weight and particularly preferably 30.0 to 50.0% by weight of at least one starch and/or starch derivative.

Particularly preferred within the fifth preferred embodiment are powdered hair care compositions according to the invention which contain starch fractions from corn, potatoes, rice, wheat, tapioca and/or dextrins, amylose, amylopectin and/or hydroxypropyl starch phosphates.

Further particularly preferred within the fifth preferred embodiment are powdered hair care compositions according to the invention which contain—based on their total weight—10.0 to 40.0% by weight, preferably 15.0 to 35.0% by weight and particularly preferably 20.0 to 30.0% by weight of bentonites.

The hair care compositions in powder form according to the invention comprise, as a third essential ingredient, 0.1 to 15.0% by weight of at least hair-conditioning active ingredient (based on the total weight of the powder).

Preferably, hair-conditioning active ingredients are used in a total amount of from 0.2 to 12.5% by weight, more preferably from 0.3 to 10% by weight and in particular from 0.5 to 7.5% by weight in the powder (based on the total weight of the powder).

For the purposes of the present invention, suitable hair-conditioning active ingredients are preferably understood to mean
Refatting agents and/or lipids,
Emollients,
Moisturiser,
Vitamins, vitamin derivatives and/or vitamin precursors,
cationic polymers and/or
Anti-dandruff agents understood.

Suitable refatting agents and/or lipids in the sense of the present invention are understood to be all fats and fat-like substances which preferably melt in the range of 30-150° C. These include triglycerides, mono- and/or diglycerides, waxes, fatty and wax alcohols, fatty acids, esters and/or ethers of fatty alcohols and fatty acids as well as fatty acid amides, hydrocarbons, lipoproteins, glycolipids, phospholipids or any mixtures of these substances.

Triglycerides are understood to be “natural” oils, preferably vegetable oils.

These preferably include triglycerides and mixtures of triglycerides such as amaranth seed oil, apricot kernel oil, argan oil, avocado oil, babassu oil, bamboo oil, baobab oil, canola oil, safflower oil, pomegranate seed oil, rosehip seed oil, hemp oil, hazelnut oil, red currant oil, black currant oil, jojoba oil, coconut oil, pumpkin seed oil, Macadamia nut oil, mallow oil, (sweet) almond oil, mango kernel oil, evening primrose oil, olive oil, Brazil nut oil, passion fruit oil, palm kernel oil, peach kernel oil, rambutan oil, rice bran oil, Castor Oil, Safflower Oil, Sasanqua Oil, Sesame Oil, Soybean Oil, Sunflower Oil, Tea Tree Oil, Grape Seed Oil, Tsubaki Oil, Walnut Oil, Wheat Germ Oil and/or Meadowfoam Oil.

Suitable plant butters preferably include shea butter, mango butter, murumuru butter, cocoa butter, apricot kernel butter, mafuri butter, bacuri butter, tucuma butter, ucuuba butter and/or cupuacu butter.

Waxes are natural or artificially obtained substances with the following properties: they are of solid to brittle-hard consistency, coarse to fine crystalline, translucent to cloudy, and melt above 30° C. without decomposition. They are already low viscosity and non-fibrous a little above the melting point and show a strongly temperature-dependent consistency and solubility. Natural vegetable waxes with a melting point of 30-150° C., for example, can be used according to the invention, such as e.g. Candelilla wax, carnauba wax, Japan wax, esparto grass wax, cork wax, guaruma wax, rice germ oil wax, sugar cane wax, ouricury wax, montan wax, sunflower wax, fruit waxes such as orange waxes, lemon waxes, grapefruit wax, bayberry wax and animal waxes such as beeswax, shellac wax, spermaceti, lanolin and brushing fat. In the spirit of the invention, it may be advantageous to use hydrogenated or cured waxes. Natural waxes that can be used according to the invention also include mineral waxes, such as ceresin and ozokerite, or petrochemical waxes, such as petrolatum, paraffin waxes and micro waxes. Chemically modified waxes, in particular the hard waxes, such as montan ester waxes, sasol waxes and hydrogenated jojoba waxes, can also be used as wax components. Synthetic waxes which can be used according to the invention include, for example, waxy polyalkylene waxes and polyethylene glycol waxes.

Esters of glycerol with one or more straight-chain or branched, saturated or unsaturated C8-C30 fatty acids such as glyceryl palmitate, stearate or oleate can be used as mono- and/or diglycerides. A particularly preferred mono- and/or diglyceride is a compound known under the INCI name PCA Glyceryl Oleate, as well as glyceride mixtures, e.g. Cutina® HR (hardened castor oil) and Novata® AB (mixture of C12-C18 mono-, di- and triglycerides).

The fatty alcohols which can be used according to the invention include e.g. the unbranched C₁₄-C₅₀ fatty alcohols, in particular the C₁₂-C₃₀ fatty alcohols, which are obtained from natural fats, oils and waxes, such as lauryl alcohol, myristyl alcohol, 1-pentadecanol, cetyl alcohol, 1-heptadecanol, stearyl alcohol, 1-nonadecanol, arachidyl alcohol, 1-heneicosanol, behenyl alcohol, brassidyl alcohol, lignoceryl alcohol, ceryl alcohol or myricyl alcohol. Preferred according to the invention are unbranched, saturated and unsubstituted fatty alcohols.

However, branched, saturated or unsaturated fatty alcohols can also be used according to the invention. Also usable according to the invention are fatty alcohol cuts, such as are produced during the reduction of naturally occurring fats and oils.

Preferred fatty acids are C₁₂-C₄₀ fatty acids or mixtures thereof. These include, for example, lauric, tridecanic, myristic, pentadecanic, palmitic, margaric, stearic, nonadecanic, arachic, behenic, lignoceric, cerotinic, melissic, erucic and elaeostearic acids, as well as substituted fatty acids such as 12-hydroxystearic acid. Such as 12-hydroxystearic acid, and the amides or monoethanolamides of the fatty acids, this list being exemplary and not restrictive.

Suitable hydrocarbons are understood to be liquid or solid hydrocarbons such as petrolatum, ceresin, ozokerite, paraffins, isoparaffins and/or squalene.

Suitable glycolipids in the sense of the present invention are sugar surfactants, alkyl polyglycosides and/or ceramides.

A suitable phospholipid in the sense of the present invention is lecithin.

Refatting agents and/or lipids may be used in the hair care compositions according to the invention in powder form - based on their total weight - in amounts of 0.01 to 5% by weight, more preferably 0.05 to 4.5% by weight and particularly preferably 0.1 to 4% by weight.

Emollients suitable according to the invention are preferably waxes, wax alcohols, fatty acids and esters of fatty alcohols and fatty acids, as already described earlier in this application.

Suitable moisturisers in the sense of the present invention can preferably be selected from
Polyols such as glycerol,
Lactic acid and or derivatives and/or salts thereof,
Trimethylglycine.

They can be used in the hair care compositions of the invention in powder form—based on their total weight—in amounts of 0.01 to 4% by weight, more preferably 0.05 to 3% by weight and particularly preferably 0.1 to 2% by weight.

By suitable “vitamins, vitamin derivatives and/or vitamin precursors” is preferably meant:
Vitamin A: the group of substances known as vitamin A includes retinol (vitamin A1) and 3,4-didehydroretinol (vitamin A2). The β-carotene is the provitamin of retinol. Vitamin A components include vitamin A acid and its esters, vitamin A aldehyde and vitamin A alcohol and its esters such as palmitate and acetate.
Vitamin B: the vitamin B group or vitamin B complex includes:
   Vitamin B₁ (Thiamine)
   Vitamin B₂ (Riboflavin)
   Vitamin B₃. The compounds nicotinic acid and nicotinamide (niacinamide) are frequently listed under this designation.
   Vitamin B₅ (pantothenic acid and panthenol). Within this group, panthenol is preferred. Usable derivatives of panthenol are in particular the esters and ethers of panthenol, pantolactone and cationically derivatised panthenols. Individual representatives include panthenol triacetate, panthenol monoethyl ether and its monoacetate and cationic panthenol derivatives.
   Vitamin B₆ (pyridoxine as well as pyridoxamine and pyridoxal).
Vitamin C (ascorbic acid): use in the form of palmitic acid ester, glucosides or phosphates may be preferred. Use in combination with tocopherols may also be preferred.
Vitamin E (tocopherols, especially a-tocopherol).
Vitamin F: the term “vitamin F” usually refers to essential fatty acids, especially linoleic acid, linolenic acid and arachidonic acid.
Vitamin H: Vitamin H is the compound (3aS,4S, 6aR)-2-oxohexahydrothienol[3,4-d]-imidazole-4-valeric acid, for which the trivial name biotin has since become established.

Particularly preferred are vitamins, provitamins and vitamin precursors from groups A, B, E and H. Very particularly preferred are nicotinamide, tocopherol, pantolactone and/or panthenol; especially preferred are nicotinamide, tocopherol and/or panthenol.

Vitamins, vitamin derivatives and/or vitamin precursors can be used in the hair care compositions according to the invention in powder form - based on their total weight - preferably in amounts of 0.001 to 1% by weight, more preferably 0.002 to 0.75% by weight and particularly preferably 0.005 to 0.5% by weight.

Suitable cationic polymers within the meaning of the present invention are preferably understood to be cationic polymers of natural origin, in particular cationic polysaccharide polymers. Particularly suitable examples are:
quaternised cellulose derivatives, as referred to under the names Celquat® and Polymer JR®
   are available on the market,
cationic starch derivatives, such as those commercially available under the name Mirustyle®,
hydrophobically modified cellulose derivatives, such as the cationic polymers sold under the trade name SoftCat®,
cationic alkyl polyglycosides,
cationized honey, for example the commercial product Honeyquat® 50,
cationic guar derivatives, such as in particular the products marketed under the trade names Cosmedia® Guar N-Hance® and Jaguar®.
cationic cassia and/or inulin derivatives such as those listed under the INCI designation Cassia Hydroxypropyltrimonium Chloride and Hydroxypropyltrimonium Inulin known cationic polymers.

Cationic polymers can be used in the hair care compositions of the invention in powder form—based on their total weight—preferably in amounts of 0 to 4% by weight, more preferably 0.1 to 3.0% by weight and in particular 0.15 to 2.0% by weight.

For the purposes of the present invention, suitable anti-dandruff agents include piroctone olamine, climbazole, zinc pyrithione, ketoconazole, salicylic acid, sulphur, selenium sulphide, tar preparations, undecenic acid derivatives, burdock root extracts, poplar extracts, nettle extracts, walnut shell extracts, birch extracts, willow bark extracts, rosemary extracts, arnica extracts and/or propanediol caprylates.

Preferred for the hair care compositions in powder form according to the invention are natural anti-dandruff active ingredients such as the previously mentioned plant extracts and/or anti-dandruff active ingredients accessible from natural sources such as propanediol caprylate.

The proportion by weight of anti-dandruff agents in the total weight of the powder according to the invention is preferably 0.01 to 5% by weight, more preferably 0.025 to 4% by weight and in particular 0.05 to 3% by weight.

In a particularly preferred embodiment, the hair care composition in powder form according to the invention contains at least one, preferably at least two and particularly preferably at least three hair-conditioning active ingredient(s) c) from the groups of the
Refatting agents and/or lipids,
Emollients,
Moisturiser,
Vitamins, vitamin derivatives and/or vitamin precursors,
cationic polymers and/or
Anti-dandruff active ingredients.

Within this embodiment, it is particularly preferred if the powder contains as active ingredient(s) c) lecithin, vegetable oils, butters and/or waxes, vitamins, vitamin precursors and/or vitamin derivatives, fatty acid esters and/or anti-dandruff agents.

Particularly preferred active ingredients c) are lecithin, apricot kernel oil, argan oil, avocado oil, rosehip kernel oil, hemp oil, jojoba oil, coconut oil, macadamia nut oil, (sweet) almond oil, olive oil, Brazil nut oil, peach kernel oil, castor oil, sunflower oil, grape seed oil, shea butter, Cocoa butter, PCA glyceryl oleate, carnauba wax, beeswax, ceramides, glycerin, lactic acid, trimethylglycine (betaine), nicotinamide, tocopherol, pantenol, biotin, cationic cellulose derivatives, cationic guar, cassia and/or inulin derivatives and/or propanediol caprylate.

To further increase the hair conditioning properties, protein hydrolysates and/or plant extracts may additionally be contained in the hair care composition in powder form according to the invention.

Suitable protein hydrolysates are product mixtures that can be obtained by acidic, basic or enzymatic catalysed degradation of proteins.

Protein hydrolysates of vegetable, animal and/or marine origin can be used.

Animal protein hydrolysates include elastin, collagen, keratin, silk and milk protein hydrolysates, which may also be in the form of salts. Such products are marketed under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Protein hydrolysates of vegetable origin are preferred, e.g. soy, almond, rice, pea, potato and wheat protein hydrolysates. Such products are available under the trademarks Gluadin® (Cognis), DiaMin® (Diamalt), Lexein® (Inolex) and Crotein® (Croda).

Cationized protein hydrolysates can also be used, whereby the underlying protein hydrolysate can be derived from animals, for example from collagen, milk or keratin, from plants, for example from wheat, corn, rice, potatoes, soy or almonds, from marine life forms, for example from fish collagen or algae, or from biotechnologically produced protein hydrolysates. The protein hydrolysates underlying the cationic derivatives can be obtained from the corresponding proteins by chemical, especially alkaline or acid hydrolysis, by enzymatic hydrolysis and/or a combination of both types of hydrolysis. The hydrolysis of proteins usually results in a protein hydrolysate with a molecular weight distribution of about 100 Daltons up to several thousand Daltons. Preferred are those cationic protein hydrolysates whose underlying protein fraction has a molecular weight of 100 to 25000 Dalton, preferably 250 to 5000 Dalton. Furthermore, cationic protein hydrolysates are quaternized amino acids and mixtures thereof. Quaternization of the protein hydrolysates or amino acids is often carried out using quaternary ammonium salts such as N,N-dimethyl-N-(n-alkyl)-N-(2-hydroxy-3-chloro-n-propyl)-ammonium halides. Furthermore, the cationic protein hydrolysates can also be further derivatised. Typical examples of the cationic protein hydrolysates and derivatives are the products known under the INCI designations and commercially available: Cocodimonium Hydroxypropyl Hydrolyzed Collagen, Cocodimopnium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed keratin, Hydroxypropyltrimonium Hydrolyzed Rice Bran Protein, Hydroxyproypltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein.

The percentage by weight of the protein hydrolysate(s) in the total weight of the powder is preferably 0.01 to 3% by weight, more preferably 0.025 to 2% by weight and in particular 0.05 to 1% by weight.

Suitable plant extracts are mainly the extracts of green tea, oak bark, nettle, witch hazel, hops, henna, chamomile, burdock root, horsetail, hawthorn, linden blossom, almond, Aloe vera, spruce needle, horse chestnut, date palm, cinnamon tree, sandalwood, juniper, coconut, mango, apricot, lime, wheat, kiwi, melon, orange, grapefruit, sage, rosemary, birch, mallow, Valerian, Meadowfoam, Quender, Yarrow, Thyme, Melissa, Hauhechel, Coltsfoot, Marshmallow, Meristem, Ginseng, Coffee, Cocoa, Moringa, Ginger Root and Ayurvedic plant extracts such as Aegle Marmelos (Bilwa), Cyperus Rotundus (Nagar Motha), Emblica Officinalis (Amalki), Morida Citrifolia (Ashyuka), Tinospora Cordifolia (Guduchi), Santalum album, (Chandana), Crocus Sativus (Kumkuma), Cinnamonum Zeylanicum and Nelumbo Nucifera (Kamala), sweet grasses such as wheat, barley, rye, oats, spelt, corn, the different varieties of millet (panicle millet, Finger millet, foxtail millet as examples), sugarcane, ryegrass, meadow foxtail, smooth oats, bunchgrass, meadow fescue, pipegrass, bamboo, cotton grass, lampbush grasses, Andropogonodeae (Imperata Cylindrica also called flame grass or cogon grass), buffalo grass, silt grasses, dogtooth grasses, love grasses, Cymbopogon (lemon grass), Oryzeae (rice), Zizania (wild rice), Beach oats, perennial oats, honey grasses, quaking grasses, meadow grasses, couch grasses, and Echinacea, especially Echinacea angustifolia DC, Echinacea paradoxa (Norton), Echinacea simulata, E. atrorubens, E. tennesiensis, Echinacea strigosa (Mc Gregor), Echinacea laevigata, Echinacea purpurea (L.) Moench and Echinacea pallida (Nutt), all kinds of seaweeds, coral moss and seaweed (such as Macrocystis Pyrifera extract), all kinds of vines and pericarp of Litchie chinensis.

Suitable extracts can be obtained from the fruits, seeds, flowers, roots, leaves and/or barks of the above plants or from the whole plants (algae, seaweed).

According to the invention, the plant extracts can be used both in pure and diluted form. If they are used in diluted form, they usually contain approx. 2-80% by weight of active substance and as solvent the extracting agent or extracting agent mixture used in their extraction. Suitable extraction agents are usually water and/or alcohols.

The plant extract(s) may be used in the powder preferably in total amounts of 0.001 to 1% by weight, preferably 0.0025 to 0.75% by weight, and more preferably 0.05 to 0.5% by weight, the amounts being based on the weight of the powder.

Further active ingredients, auxiliaries and additives which may preferably be contained in the powdered cosmetic hair care compositions according to the invention are, for example:
Perfumes, if necessary also in capsule form,
UV-Filter,
(natural) dyes for colouring the agent,
Active ingredients such as bisabolol and/or allantoin,
Complexing agents such as EDTA, NTA, β-alanine diacetic acid and phosphonic acids,
Antioxidants,
pH adjusting agents such as citric acid and/or lactic acid,
Salts (NaCl).

A significant advantage of the powder hair care compositions according to the invention over commercially available concentrates is that no free water needs to be added to them, which means that they have an even smaller volume and preservation of the powder is not absolutely necessary.

A small amount of water may nevertheless be present in the powdered hair care compositions of the invention as a component of commercial products, but preferably they are substantially free of water.

For the purposes of the present invention, “substantially free” of water is preferably understood to mean a maximum water content of 5% by weight, more preferably 3% by weight and particularly preferably 1.5% by weight (based on the total weight of the powdered hair care compositions according to the invention).

Similarly, it is possible that the powder hair care compositions according to the invention nevertheless comprise a small proportion of preservatives as a component of commercial products, but preferably they are substantially free of preservatives.

For the purposes of the present invention, “substantially free” of preservatives is preferably understood to mean a maximum preservative content of 1% by weight, more preferably of 0.5% by weight and particularly preferably of 0.1% by weight (based on the total weight of the powdered hair care compositions according to the invention).

In a preferred embodiment, the powdered cosmetic cleansing compositions of the invention are substantially free of water and/or preservatives.

The invention also relates to a powder cosmetic hair care composition as described above in the form of a powder which can be dissolved and/or emulsified after contact with water.

A powder form is well, namely particularly quickly, soluble. This corresponds to the spirit of the times, but at the same time is associated with a small “wellness experience” during application, as the use of the powder creates a new feeling of application that differs from previous experiences and emphasises the playful side of body care.

The invention also relates to a process for the preparation of a powder cosmetic hair care composition as previously described, in which all the ingredients are mixed and optionally sieved in a mixing apparatus for cosmetic powder products.

This has the advantage of providing a solid cosmetic hair care composition in powder form, and the sieving ensures a uniform grain size that is perceived as pleasant. The advantage of such a process is that it does not require melting at high temperatures, which saves energy and is very gentle on the ingredients. This is particularly important when using largely natural low-processed ingredients that can provide benefits not achievable with highly processed products.

The invention also relates to a method of caring for the hair, in which a cosmetic composition in powder form, as previously described, is dissolved in water, rubbed between the hands and/or emulsified, distributed on the hair and rinsed off with water after a contact time of 10 seconds to 15 minutes.

This procedure makes the advantages described above tangible for a user and thus represents an enrichment not only for personal hygiene and care but also a sensory enrichment.

The invention also relates to the use of a cosmetic composition in powder form, as previously described, for the care of the hair, in particular for improving the
Hair feel, in particular the hair feel on wet hair and/or in the lengths and/or ends of the hair,
Disentanglability and/or
Combability.

This use makes the advantages described above tangible for a user and thus represents an enrichment not only for personal hygiene and care but also a sensory enrichment.

Another novel and exciting form of application of the powder compositions according to the invention is packaging, for example in a (paper) sachet, a jar and/or in recycled plastic material. In addition to the new sensory experience, this is accompanied above all by the advantage of a relatively fast resolution due to a high surface area. The packaging materials glass and paper are easily recyclable packaging materials that are popular with environmentally conscious buyers, and their properties are therefore well suited to the properties of the powdered cosmetic compositions described and claimed here.

### EXAMPLES

Basic exemplary compositions are shown in the following tables 1-7 (compositions Z1 to Z25). Quantities refer to [% by weight] (unless otherwise stated):

TABLE 1

| Ingredient | Z1 | Z2 | Z3 | Z4 |
|---|---|---|---|---|
| (a): one or more surfactant(s) | 5-35 | 6-34 | 7-33 | 8-32 |
| b): Binder and/or thickener | 40-80 | 45-80 | 50-75 | 55-75 |
| (c): one or more hair | 0.1-15 | 0.2-12.5 | 0.3-10 | 0.5-7.5 |
| conditioning active substance(s) | | | | |
| If necessary, other auxiliaries | ad 100 | ad 100 | ad 100 | ad 100 |
| and additives | | | | |

TABLE 2

| Ingredient | Z5 | Z6 | Z7 | Z8 |
|---|---|---|---|---|
| (a): one or more anionic | 5-35 | 6-34 | 7-33 | 8-32 |
| surfactant(s) | | | | |
| b): Binder and/or thickener | 40-80 | 45-80 | 50-75 | 55-75 |
| (c): one or more hair conditioning | 0.1-15 | 0.2-12.5 | 0.3-10 | 0.5-7.5 |
| active substance(s) | | | | |
| If necessary, other auxiliaries and | ad 100 | ad 100 | ad 100 | ad 100 |
| additives | | | | |

TABLE 3

| Ingredient | Z9 | Z10 | Z11 | Z12 |
|---|---|---|---|---|
| (a): one or more anionic | 5-35 | 6-34 | 7-33 | 8-32 |
| surfactant(s) | | | | |
| b): Binder and/or thickener | 40-80 | 45-80 | 50-75 | 55-75 |
| (c): one or more hair conditioning | 0.1-15 | 0.2-12.5 | 0.3-10 | 0.5-7.5 |
| active substance(s) | | | | |
| one or more non-ionic, cationic, | 0-10 | 0.1-8 | 0.2-6 | 0.3-5 |
| amphoteric and/or zwitterionic | | | | |
| surfactants | | | | |
| If necessary, other auxiliaries and | ad 100 | ad 100 | ad 100 | ad 100 |
| additives | | | | |

TABLE 4

| Ingredient | Z13 | Z14 | Z15 | Z16 |
|---|---|---|---|---|
| a): Amino acid-based surfactants, | 5-35 | 6-34 | 7-33 | 8-32 |
| isethionates and/or alkyl sulphates | | | | |
| b): Binder and/or thickener | 40-80 | 45-80 | 50-75 | 55-75 |
| (c): one or more hair conditioning | 0.1-15 | 0.2-12.5 | 0.3-10 | 0.5-7.5 |
| active substance(s) | | | | |
| If necessary, other auxiliaries and | ad 100 | ad 100 | ad 100 | ad 100 |
| additives | | | | |

TABLE 5

| Ingredient | Z17 | Z18 | Z19 | Z20 |
|---|---|---|---|---|
| a): Amino acid-based surfactants, | 5-35 | 6-34 | 7-33 | 8-32 |
| isethionates and/or alkyl sulphates | | | | |
| b1): Starch and/or starch derivative | 20-60 | 25-55 | 30-50 | 35-50 |
| b2): Bentonite | 10-40 | 15-35 | 17-33 | 20-30 |
| (c): one or more hair conditioning | 0.1-15 | 0.2-12.5 | 0.3-10 | 0.5-7.5 |
| active substance(s) | | | | |
| If necessary, other auxiliaries and | ad 100 | ad 100 | ad 100 | ad 100 |
| additives | | | | |

TABLE 6

| Ingredient | Z21 | Z22 | Z23 | Z24 |
|---|---|---|---|---|
| a): Amino acid-based surfactants, | 5-35 | 6-34 | 7-33 | 8-32 |
| isethionates and/or alkyl sulphates | | | | |
| b1): Starch and/or a starch derivative | 20-60 | 25-55 | 30-50 | 35-50 |
| b2): Bentonite | 10-40 | 15-35 | 17-33 | 20-30 |
| Lipids, vegetable oils, butters and/or | 0.1-15 | 0.2-12.5 | 0.3-10 | 0.5-7.5 |
| waxes, protein hydrolysates, | | | | |
| silicones, plant extracts and/or anti- | | | | |
| dandruff active ingredients | | | | |
| If necessary, other auxiliaries and | ad 100 | ad 100 | ad 100 | ad 100 |
| additives | | | | |

TABLE 7

| Ingredient | Z25 |
|---|---|
| Sodium Cocoyl Isethionate, | 20-32 |
| (Di)sodium Cocoyl Glutamate | |
| Maize starch (ZEA Mays (Corn) Starch) | 35-50 |
| and/or maltodextrin | |
| Bentonite | 10-30 |
| Tocopherol | 0-0.5 |
| Lecithin | 0-7.5 |
| Glycerine | 0-5 |
| If necessary, other auxiliaries and additives | ad 100 |

What is claimed is:

1. A cosmetic care composition in powder form for keratinous fibres, comprising, by weight of the total composition
a) 5.0 to 35.0% by weight of at least one surfactant,
b) 40.0 to 80.0% by weight of at least one binder and/or thickener, and
c) 0.1 to 15.0% by weight of at least one hair conditioning active ingredient.

2. The cosmetic care composition in powder form according to claim 1, wherein the surfactant(s) comprise anionic surfactants and are selected from the group consisting of amino-acid based surfactants, isethionates, alkyl sulphates, and combinations thereof.

3. The cosmetic care composition in powder form according to claim 1, wherein the surfactant(s) a) comprise anionic, non-ionic, cationic, amphoteric and/or zwitterionic surfactants.

4. The cosmetic care composition in powder form according to claim 1, wherein the binders and/or thickeners b) are of vegetable and/or mineral origin in a proportion by weight of at least 95%, based on the total weight of all binders and/or thickeners in the powder.

5. The cosmetic care composition in powder form according to claim 1, wherein the binder and/or thickener b) are selected from the group consisting of clay minerals, polysaccharides, bentonites, starches, starch derivatives, and combinations thereof.

6. The cosmetic care composition in powder form according to claim 5, comprising 20.0 to 60.0% by weight of at least one starch and/or starch derivative, and/or 10.0 to 40.0% by weight of bentonites.

7. The cosmetic care composition in powder form according to claim 1, wherein the at least one hair-conditioning active ingredient(s) c) is selected from the group consisting of
Refatting agents and/or lipids,
Emollients,
Moisturiser,
Vitamins, vitamin derivatives and/or vitamin precursors,
cationic polymers,
Anti-dandruff active ingredients, and combinations thereof.

8. The cosmetic care composition in powder form according to claim 1, which is dissolved and/or emulsified in contact with water.

9. A cosmetic process for the care of hair comprising obtaining the cosmetic care composition in powder form according to claim 1 and dissolving in water, rubbing between the hands, and/or spreading on the hair in an emulsified form and rinsing off with water after a contact time of 10 seconds to 15 minutes.

10. The cosmetic care composition in powder form according to claim 5, wherein the at least one starch and/or starch derivative is 25.0 to 55.0% by weight and/or the bentonites is 15.0 to 35.0% by weight.

11. The cosmetic care composition in powder form according to claim 5, wherein the at least one starch and/or starch derivative is 30.0 to 50.0% by weight of and/or the bentonites is 10.0 to 30.0% by weight.

12. The cosmetic care composition in powder form according to claim 7, comprising at least two hair-conditioning active ingredient(s) from c).

13. The cosmetic care composition in powder form according to claim 7, comprising at least three hair-conditioning active ingredient(s) from c).
